(19) United States
(12) Patent Application Publication
(10) Pub. No.: US 2025/0271415 A1
(43) Pub. Date: Aug. 28, 2025

(54) BIOPARTICLE ANALYSIS SYSTEM, INFORMATION PROCESSING DEVICE, AND BIOPARTICLE ANALYSIS METHOD

(71) Applicant: Sony Group Corporation, Tokyo (JP)

(72) Inventor: Masahiro Matsumoto, Kanagawa (JP)

(73) Assignee: Sony Group Corporation, Tokyo (JP)

(21) Appl. No.: 18/847,823

(22) PCT Filed: Feb. 14, 2023
(86) PCT No.: PCT/JP2023/004939

§ 371 Date: May 13, 2025

(30) Foreign Application Priority Data
Mar. 29, 2022 (JP) 2022-053901

(51) Int. Cl. G01N 33/483; G01N 33/53

(52) U.S. Cl. CPC G01N 33/483; G01N 33/53

(57) ABSTRACT

Provided is a technique capable of associating morphological information with molecular information with high accuracy.

The present technology provides a bioparticle analysis system, and the like, including: a capturing device that captures a bioparticle via a bioparticle capturing part on a surface on which a cleavable linker, the bioparticle capturing part, a molecule capturing sequence part, and a barcode sequence part are immobilized via the linker; and an information processing device that associates morphological information regarding the bioparticle obtained on the basis of captured image information with information regarding a molecule obtained on the basis of a barcode sequence part assigned to the molecule derived from the bioparticle captured by the molecule capturing sequence part.

### TECHNICAL FIELD

The present technology relates to a bioparticle analysis system, an information processing device, and a bioparticle analysis method. More specifically, the present invention relates to a bioparticle analysis system, an information processing device, and a bioparticle analysis method capable of associating morphological information with molecular information with high accuracy.

### BACKGROUND ART

In related art, various methods for performing single cell analysis have been proposed. For example, Non-Patent Document 1 discloses a method in which mobility of a cell is analyzed by, after creating patterning by light irradiation using a substrate to which a cell membrane-binding molecule is bound via a photodegradable linker, performing cell seeding to trap a single cell.

### CITATION LIST

### Non-Patent Document

Non-Patent Document 1: Collagen Surfaces Modified with Photo-Cleavable Polyethylene Glycol-Lipid Support Versatile Single-Cell Arrays of Both Non-adherent and Adherent Cells, Macro Molecular Bioscience, 1670-6, (2014)

### SUMMARY OF THE INVENTION

### Problems to be Solved by the Invention

However, in related art, there is a problem that there is no method for associating morphological information with molecular information with high accuracy in single cell analysis, or the like.

On the other hand, in order to integrate the morphological information and the molecular information at a single cell level on a one-to-one basis, while a method using a 96 well or 384 well plate is conceivable, the number of cells that can be analyzed by one plate is about several hundred, and it takes high cost to perform sequencing for each bioparticle/well.

Furthermore, in a case of using a plate having thousands to tens of thousands of microwells, barcoded molecules of all the wells can be collectively sequenced through a step of performing imaging after encapsulating one bioparticle in each well, encapsulating one barcode bead or barcode gel in each well, dissolving one bioparticle in the well and capturing molecules with the barcode bead to give a barcode, which can reduce analysis cost. However, the barcode beads are randomly encapsulated in the wells, and thus, it is difficult to connect the well and the barcode information. It is therefore difficult to connect captured image information and molecular information for each well.

Thus, a main object of the present technology is to provide a technology capable of associating morphological information with molecular information with high accuracy.

### Solutions to Problems

The present technology first provides a bioparticle analysis system including: a capturing device that captures a bioparticle via a bioparticle capturing part on a surface on which a cleavable linker, the bioparticle capturing part, a molecule capturing sequence part, and a barcode sequence part are immobilized via the linker; and an information processing device that associates morphological information regarding the bioparticle obtained on the basis of captured image information with information regarding a molecule obtained on the basis of the barcode sequence part assigned to the molecule derived from the bioparticle captured by the molecule capturing sequence part.

The present technology also provides an information processing device that associates morphological information regarding a bioparticle obtained on the basis of captured image information with information regarding a molecule obtained on the basis of a barcode sequence part assigned to the molecule derived from the bioparticle.

The present technology further provides a bioparticle analysis method including: a capturing step of capturing a bioparticle via a bioparticle capturing part on a surface on which a cleavable linker, the bioparticle capturing part, a molecule capturing sequence part, and a barcode sequence part are immobilized via the linker; an imaging step of imaging the bioparticle captured on the surface; a sequence analysis step of analyzing a sequence of the barcode sequence part assigned to a molecule derived from the bioparticle captured by the molecule capturing sequence part; and an association step of associating morphological information regarding the bioparticle obtained on the basis of captured image information obtained in the imaging step with information regarding the molecule obtained on the basis of the sequence of the barcode sequence part obtained in the sequence analysis step.

### BRIEF DESCRIPTION OF DRAWINGS

FIG. 1 is a schematic view illustrating one example of an embodiment of a bioparticle analysis system 100 according to a first embodiment.

FIG. 2 is a schematic view illustrating one example of an embodiment of a capturing device 1.

FIG. 3 is a schematic view illustrating one example of an embodiment of the capturing device 1 different from FIG. 2.

FIG. 4 is a schematic view illustrating one example of an embodiment of the bioparticle analysis system 100 according to a second embodiment.

FIG. 5 is a schematic view illustrating one example of an embodiment of the bioparticle analysis system 100 according to a third embodiment.

FIG. 6 is a flowchart for explaining a first flow example.

FIG. 7 is a schematic view for explaining a particle isolation step.

FIG. 8 is a schematic view for explaining the particle isolation step.

FIG. 9 is a schematic view illustrating one example of an embodiment of a microchannel to be used in the particle isolation step.

FIG. 10 is a schematic view illustrating one example of an embodiment of a nucleic acid-binding antibody.

FIG. 11 is a view schematically illustrating one example of an embodiment of a bioparticle sorting device to be used in the particle isolation step.

FIG. 12 is a flowchart for explaining a second flow example.

FIG. 13 is a flowchart for explaining a third flow example.

FIG. 14 is a conceptual diagram for explaining an inference step S12.

FIG. 15 is a schematic view for explaining operation in each step included in a bioparticle analysis method according to a fourth embodiment.

### MODE FOR CARRYING OUT THE INVENTION

Hereinafter, preferred modes for carrying out the present technology will be described with reference to the drawings. Embodiments to be described hereinafter illustrate examples of representative embodiments of the present technology, and any embodiments can be combined. Furthermore, the scope of the present technology is not narrowly construed on the basis of these. Note that the description will be given in the following order.
1. First embodiment (bioparticle analysis system 100)
(1) Overall configuration
(2) Capturing device 1
(2-1) Linker 11
(2-2) Amplification sequence part 12
(2-3) Barcode sequence part 13
(2-4) Unique molecular identifier (UMI) part 14
(2-5) Molecule capturing sequence part 15
(2-6) Bioparticle capturing part 16
(2-7) Collection sequence part 17
(3) Information processing measure 2
(3-1) Processing unit 21
(3-2) Storage unit 22
(3-3) User interface unit 23
(3-4) Output unit 24
(4) Imaging device 3
2. Second embodiment (bioparticle analysis system 100)
(1) Overall configuration
(2) Fluid control unit 4
3. Third embodiment (bioparticle analysis system 100)
(1) Overall configuration
(2) Microchip 150
4. Fourth embodiment (bioparticle analysis method)
(1) Overall configuration
(2) First flow example
(2-1) Preparation step S1
(2-2) Capturing step S2
(2-3) Imaging step S3
(2-4) Sequence analysis step S4
(2-5) Association step S5
(2-6) Cleavage step S6
(2-7) Isolation step S7
(2-7-1) Discrimination step
(2-7-2) Particle isolation step
(2-7-2-1) In case of space in well
(2-7-2-2) In case of space in emulsion particle
(2-8) Breakage step S8
(2-9) Target molecule analysis step S9
(3) Second flow example
(3-1) Stimulation step S10
(4) Third flow example
(4-1) Learned model creation step S11
(4-2) Inference step S12

### 1. First Embodiment (Bioparticle Analysis System 100)

### (1) Overall Configuration

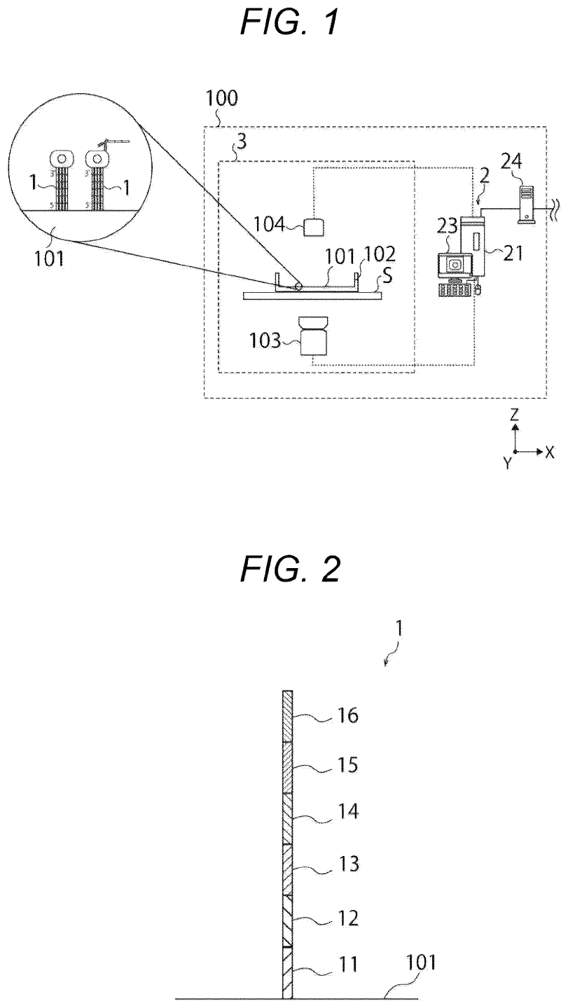

An overall configuration of a bioparticle analysis system 100 according to a first embodiment of the present technology will be described with reference to FIG. 1. The bioparticle analysis system 100 according to the present embodiment includes a capturing device 1, an information processing device 2, and an imaging device 3. Furthermore, other devices and portions may also be provided as needed.

Hereinafter, each device and each portion will be described in detail.

### (2) Capturing Device 1

FIG. 2 is a schematic view illustrating one example of an embodiment of the capturing device 1. The capturing device 1 is a portion that captures a bioparticle via a bioparticle capturing part 16 on a surface 101 on which a cleavable linker 11, the bioparticle capturing part 16, a molecule capturing sequence part 15, and a barcode sequence part 13 are immobilized via the linker 11.

In the present specification, the “bioparticle” may include a chromosome, a ribosome, a mitochondrion, an organelle (organelle), and the like, constituting various cells. The cell may include an animal cell (for example, a blood cell, or the like), a plant cell. The cell may in particular be a blood-derived cell or a tissue-derived cell. Furthermore, the cell may include a floating cell. The blood cell may be, for example, a suspension cell such as a T cell and a B cell. The tissue-derived cell may be, for example, an adherent cell separated from an adherent cultured cell or a tissue, or the like. A mass of cells may include, for example, spheroid, organoid, and the like. A microorganism may include bacteria such as Escherichia coli, viruses such as a tobacco mosaic virus, fungi such as yeast, and the like. Furthermore, the bioparticle can also include a biological polymer such as a nucleic acid, protein, and a complex thereof. The biological polymer may be, for example, one extracted from a cell, or one contained in a blood sample or other liquid samples.

In the present technology, the bioparticle is preferably a cell or a mass of cells. Examples of the mass of cells include spheroid and organoid. A barcode sequence binds to the mass of cells on an analysis substrate 102 described later for each mass of cells. Thereafter, by performing cleavage, isolation, and breakage, a unique barcode sequence can be assigned to each mass of cells. As a result, captured image information and morphological information for each mass of cells are associated with information on the molecule derived from the bioparticle.

In the present technology, the bioparticle may be stimulated by a drug. In the present specification, the “drug” means a chemical substance that kills a pathogenic microorganism such as a bacterium and a virus and a cancer cell (malignant neoplasm) or suppresses proliferation thereof, a chemical substance that acts on a blood cell such as a T cell and a B cell, and the like, and is not particularly limited in the present technology. In addition, the “drug” is a broad concept including drug candidates in a development stage.

Hereinafter, one example of a structure of the capturing device 1 will be described in detail with reference to FIG. 2. The capturing device 1 illustrated in FIG. 2 includes the linker 11, an amplification sequence part 12, the barcode sequence part 13, a unique molecular identifier (UMI) part 14, the molecule capturing sequence part 15, and the bioparticle capturing part 16. The capturing device 1 is immobilized on the surface 101 via the linker 11.

For example, as illustrated in FIG. 2, the capturing device 1 can be prepared on the surface 101 of the analysis substrate 102 such as a glass slide. The capturing device 1 may be, for example, either a single molecule or a complex molecule, and the single molecule may mean, for example, one type of molecule having a plurality of functions, and may be one nucleic acid (for example, DNA or RNA) containing each portion configured as the linker 11. The complex molecule may be, for example, a molecular assembly (for example, a conjugate of two or more kinds of molecules, and the like) including two or more kinds of molecules, or may be a conjugate of a nucleic acid and polypeptide (such as, for example, protein or part thereof and oligopeptide).

Note that the amplification sequence part 12, the barcode sequence part 13, and the UMI part 14 to be described later may be configured as a continuous nucleic acid (in particular, DNA). In a case where the bioparticle capturing part 16 is a nucleic acid, in addition to these, the molecule capturing sequence part 15 may also be configured as a continuous nucleic acid (in particular, DNA). In these cases, for example, an end close to a portion where the capturing device 1 is immobilized on the surface 101 may be an end 5′, and the other end may be an end 3′.

### (2-1) Linker 11

The linker 11 may be a linker cleavable by stimulation, and may be, for example, a linker cleavable by light stimulation or chemical stimulation. The light stimulation is suitable because it can selectively stimulate specific locations.

The linker 11 may contain, for example, as a linker cleavable by light stimulation, any one or more groups selected from a group including an arylcarbonylmethyl group, a nitroaryl group, a coumarin-4 ylmethyl group, an arylmethyl group, a metal-containing group, and other groups known in related art.

Examples of the arylcarbonylmethyl group include a phenacyl group, an o-alkylphenacyl group, and a p-hydroxyphenacyl group.

Examples of the nitroaryl group include an o-nitrobenzyl group, an o-nitro-2 phenethyloxycarbonyl group, and o-nitroanilide.

Examples of the arylmethyl group include one into which a hydroxy group is introduced, and one into which a hydroxy group is not introduced.

In a case where the linker 11 is a linker cleavable by light stimulation, the linker 11 may be cleaved by light having a wavelength of preferably 360 nm or more. The linker 11 may be a linker that is preferably cleaved at an energy of 0.5 μJ/μm² or less. By adopting a linker cleaved by light having the above wavelength or the above energy, it is possible to reduce cell damage (particularly, cleavage of DNA or RNA, and the like) that may occur when a light stimulus is applied.

Preferably, the linker 11 may be a linker cleaved by light in a short wavelength range, specifically light in a wavelength range of 360 nm to 410 nm, or may be a linker cleaved by light in a near infrared region or an infrared region, specifically light in a wavelength range of 800 nm or more. In a case where the linker 11 is a linker that is efficiently cleaved by light having a wavelength in a visible light region, it may be difficult to handle the analysis surface. Thus, the linker 11 is preferably a linker cleaved by the light in the short wavelength range, or the light in the near infrared region or the infrared region.

Furthermore, the linker 11 may include, for example, a disulfide bond, a restriction endonuclease recognition sequence, a sequence complementary to guide RNA (gRNA), an RNA sequence, and the like, as a linker cleavable by chemical stimulation. For cleavage of the disulfide bond, for example, a reducing agent such as Tris (2-carboxyethyl) phosphine (TCEP), Dithiothreitol (DTT), and 2-Mercaptoethanol is used. For example, in a case where TCEP is used, reaction is performed using 50 mM of TCEP for about 15 minutes. For dissociation of the restriction endonuclease recognition sequence, an appropriate restriction enzyme is used according to each sequence. 1U of restriction endonuclease activity is an amount of enzyme that completely degrades 1 μg of ADNA per hour at 37° C. in principle in 50 μL of each enzyme reaction solution, and it is only necessary to adjust the amount of enzyme according to an amount of the restriction endonuclease recognition sequence. In a case where gRNA is used, CRISPR associated (Cas) nuclease enables dissociation of a gRNA complementary sequence part. Depending on the type of Cas nuclease, the linker 11 may include a protospacer adjacent motif (PAM) sequence. In this case, the PAM sequence is adjacent to the sequence complementary to the gRNA. In a case where the RNA sequence is included, the RNA sequence part is dissociated by treatment with RNase.

Furthermore, the linker 11 may include a plurality of cleavable linkers in the capturing device 1 in order to increase cleavage efficiency. Preferably, the plurality of linkers 11 may be connected in series. For example, in a case where a cleavage probability of one linker is 0.8, the cleavage probability is improved to 0.992 (=1−0.2³) by connecting three linkers in series.

### (2-2) Amplification Sequence Part 12

The amplification sequence part 12 can include, for example, a nucleic acid having a primer sequence to be used for amplification of a nucleic acid or a promoter sequence to be used for transcription of a nucleic acid in a target molecule analysis step S9 described later. The nucleic acid may be DNA or RNA and is particularly DNA. The amplification sequence part 12 may have both the primer sequence and the promoter sequence. The primer sequence may be, for example, a PCR handle. The promoter sequence may be, for example, a T7 promoter sequence.

### (2-3) Barcode Sequence Part 13

The barcode sequence part 13 includes a nucleic acid having a barcode sequence. The nucleic acid may be particularly DNA or RNA, and more particularly DNA. The barcode sequence may be used, for example, to specify a captured bioparticle (in particular, a cell or an exosome), and in particular, may be used as an identifier to make a bioparticle isolated in a certain microspace distinguishable from a bioparticle isolated in another microspace. Furthermore, the barcode sequence can be used as an identifier for making the capturing device 1 including a certain barcode sequence distinguishable from the capturing device 1 including another barcode sequence. The barcode sequence may be associated with a bioparticle to which the capturing device 1 including the barcode sequence binds. Furthermore, the barcode sequence may be associated with a microspace in which the bioparticle to which the capturing device 1 including the barcode sequence binds is isolated, and in particular, may be associated with information regarding a position of the microspace (hereinafter, also referred to as “position information”). The position information may be information for specifying a position on the surface 101, and is, for example, information regarding XY coordinates, but the present technology is not limited thereto.

Furthermore, in the present technology, the barcode sequence is associated with morphological information regarding the bioparticle obtained on the basis of captured image information. In the present specification, the “captured image information” may be data itself of a captured image, but the present technology is not limited thereto. For example, the “captured image information” may be data obtained by compressing the captured image. Furthermore, the “morphological information” includes the captured image itself, a feature amount extracted from the captured image, and the like, and is a broad concept including one-dimensional, two-dimensional, and three-dimensional information. Examples of the feature amount include a radius (mean of distances from center to points on the perimeter), texture (standard deviation of gray-scale values), a perimeter, an area, smoothness (local variation in radius lengths), compactness (perimeter\hat{ }2/area−1.0), concavity (severity of concave portions of the contour), concave points (number of concave portions of the contour), symmetry, a fractal dimension (coastline approximation−1), roundness, mean intensity, max intensity, speckles within a nucleus, distances between the nucleus and individual cytoplasmic vesicles, but the present technology is not limited thereto. Furthermore, for example, a convolutional neural network, or the like, can be used to extract a feature amount other than those described above from the captured image. Note that the feature amount may be extracted by the information processing device 2 described later.

Furthermore, an ID number may be assigned to the captured image itself and the barcode sequence associated with the feature amount extracted from the captured image. The ID number may be used in steps subsequent to the cleavage step S6. The ID number may correspond to the barcode sequence on a one-to-one basis and may be used as data corresponding to the barcode sequence in steps subsequent to a cleavage step S6.

In the present technology, a plurality of capturing devices 1 immobilized in a certain region of the surface 101 may have the same barcode sequence. As a result, the certain region is associated with the barcode sequence. By setting a size of the certain region to be smaller than a size of the bioparticle, the capturing device 1 including the barcode sequence can be associated with a position where one bioparticle exists. For example, a region R in which the plurality of capturing devices 1 including the same barcode sequence is immobilized may be smaller than the size of the bioparticle.

The surface 101 to be used in the bioparticle analysis system 100 according to the present technology may have a plurality of regions in which a plurality of the capturing devices 1 having the same barcode sequence is immobilized. The barcode sequence may be different for each region. The size (for example, a maximum dimension of the region, a diameter, a long diameter, a length of the long side, or the like, of the region) of each region may be preferably smaller than the size of the bioparticle. The size of each region may be, for example, 50 μm or less, preferably 10 μm or less, and more preferably 5 μm or less. The plurality of regions can be arranged at intervals such that, for example, the bioparticle captured in one region is not captured by the capturing device 1 immobilized in another region. The interval may be, for example, a distance equal to or greater than the size of the bioparticle and may be preferably a distance greater than the size of the bioparticle. The number of the plurality of regions is preferably larger than the number of bioparticles applied to the surface 101 in a capturing step S2 described later. This prevents two bioparticles from being captured in one region.

In one embodiment of the present technology, the capturing device 1 including a barcode sequence whose sequence is known can be immobilized in a predetermined region. For example, the surface 101 has a plurality of regions, and the plurality of capturing devices 1 respectively immobilized in the plurality of regions may include the same barcode sequence. The plurality of regions may be set to be smaller than the size of the bioparticle to be captured. With the surface 101 having such a configuration, the plurality of regions can be respectively associated with the barcode sequences included in the plurality of capturing devices 1 immobilized in the respective regions. In the present specification, a region where the capturing device 1 including the same barcode sequence is immobilized is also referred to as a “spot”. A size of the spot may be, for example, 50 μm or less, preferably 10 μm or less, and more preferably 5 μm or less.

The surface 101 configured as described above can associate the barcode sequence included in a certain capturing device 1 with the position where the certain capturing device 1 exists at the time when the capturing device 1 is immobilized on the surface 101. For the immobilization, for example, biotin is bound to the linker 11 of the capturing device 1, streptavidin is bound to the surface 101 on which the capturing device 1 is immobilized, and the biotin binds to the streptavidin, whereby the capturing device 1 is immobilized on the surface 101.

In another embodiment of the present technology, the capturing device 1 including the barcode sequence may be randomly arranged on the surface 101. In this case, after the capturing device 1 including the barcode sequence is immobilized on the surface 101, by reading the barcode sequence included in the immobilized capturing device 1, the barcode sequence included in a certain capturing device 1 is associated with the position where the certain capturing device 1 exists.

In addition, the barcode sequence included in a certain capturing device 1 does not have to be associated with the position where the certain capturing device 1 exists. The bioparticle and the capturing device 1 are isolated in the microspace by the isolation step S7 described later, and thus, the bioparticle is associated with the capturing device 1 (in particular, the barcode sequence included in the capturing device 1) on a one-to-one basis. In this case, for example, beads (for example, gel beads) to which a plurality of capturing devices 1 including the same barcode sequence bind may be used, and the beads can be immobilized on the surface 101. A size of the bead may be, for example, 50 μm or less, preferably 10 μm or less, and more preferably 5 μm or less. In order to bind the capturing device 1 to the bead, for example, a combination of biotin and streptavidin may be used. For example, biotin is bound to the linker 11 of the capturing device 1, streptavidin is bound to the bead, and the biotin binds to the streptavidin, whereby the capturing device 1 is immobilized to the bead.

The surface 101 may be provided with a plurality of recesses. One spot or one bead described above may be arranged in each of the plurality of recesses. With the plurality of recesses, the spots or the beads can be more easily arranged on the surface 101. A size of the recess is preferably a size in which one bead is put, for example. A shape of the recess may be a circle, an ellipse, a hexagon, or a quadrangle, but the present technology is not limited thereto.

Furthermore, in the surface 101, a surface state of a surface portion on which the spot or the bead is arranged may be different from surface states of other surface portions. For example, the surface portion on which the spot or the bead is arranged may be hydrophilic, and other surface portions may be hydrophobic, or other surface portions may be hydrophobic and have protrusions. Examples of a method for imparting hydrophilicity to the surface include reactive ion etching in the presence of oxygen, irradiation with deep ultraviolet light in the presence of ozone, and the like. In these methods, a mask having a penetrated portion corresponding to a portion to which hydrophilicity is to be imparted may be used. In addition, examples of a method for imparting hydrophobicity to the surface include silicone spray (spray-on-silicone), and for example, Techspray 2101-12S, or the like, may be used. Even in a case of imparting hydrophobicity, for example, a mask having a penetrated portion corresponding to a portion to which hydrophobicity is to be imparted may be used.

In addition, for example, the capturing device 1 can be synthesized on the substrate 102 using a DNA microarray preparation technique, an oligopool synthesis technique, or the like. For example, the capturing device 1 can be synthesized at a specific position using a technique such as a digital mircomirror device (DMD) to be used for photolithography, a liquid crystal shutter, or a spatial light phase modulator. Alternatively, a base or oligonucleotide is electrically induced to bind to a specific location. Alternatively, a method in which synthesis is performed while a protective group of a base at a specific location is electrochemically removed, can be performed. Methods for the synthesis are described, for example, in Basic Concepts of Microarrays and Potential Applications in Clinical Microbiology, CLINICAL MICROBIOLOGY REVIEWS, October 2009, page. 611 to 633. Note that, in a case where the capturing device 1 is synthesized on the substrate 102 by the synthesis, information on the position where the capturing device 1 is synthesized is acquired at the time of synthesis, and the barcode sequence is associated with the position information. In this event, an ID number may be given.

In one embodiment of the present technology, any of the capturing devices 1 immobilized on the surface may include a common oligo sequence. By using a fluorescently labeled nucleic acid having a sequence complementary to the oligo sequence, it is possible to confirm the position (in particular, the position of the spot or the position of the bead) where the capturing device 1 is immobilized, in particular, in a dark field. In addition, in a case where there is no recess or protrusion described above on the surface, it may be difficult to grasp the position where the capturing device 1 is immobilized. In this case, the fluorescent label makes it easy to grasp the position where the capturing device 1 is immobilized.

### (2-4) UMI Part 14

The UMI part 14 may contain a nucleic acid, particularly DNA or RNA, and more particularly DNA. The UMI part 14 may have, for example, a sequence of 5 bases to 30 bases, particularly 6 bases to 20 bases, and more particularly 7 bases to 15 bases. The UMI part 14 may be configured to have different sequences between molecules derived from the bioparticles immobilized on the surface 101. For example, in a case where the UMI part 14 has a nucleic acid sequence of 10 bases, a type of the UMI sequence is the tenth power of 4, that is, one million or more.

In addition, the UMI part 14 can be used to quantify the molecule derived from the bioparticle. For example, in a case where the molecule is mRNA, a UMI sequence may be added to cDNA obtained by reverse transcription of mRNA that is the molecule. Multiple CDNAs obtained by amplifying cDNA reverse transcribed from one mRNA molecule have the same UMI sequence, but multiple cDNAs obtained by amplifying cDNA transcribed from another mRNA molecule having the same sequence as the sequence of the one mRNA have different UMI sequences. Thus, the number of copies of mRNA can be determined by counting the number of types of UMI sequences having the same cDNA sequence.

The UMI part 14 can be configured to have different sequences between molecules derived from a plurality of bioparticles including the same barcode sequence immobilized in one region R (for example, the spot or the bead), for example. In other words, the molecules derived from the plurality of bioparticles immobilized in the region R (for example, the spot or the bead) may have the same barcode sequence but different UMIs from each other.

### (2-5) Molecule Capturing Sequence Part 15

The molecule capturing sequence part 15 includes a component for capturing a molecule (hereinafter, also referred to as a “target molecule”) derived from the bioparticle captured via the bioparticle capturing part 16 described later. The component can be, for example, a nucleic acid or protein. In a case where the component is a nucleic acid, the nucleic acid may be, for example, a polyT sequence in order to comprehensively capture mRNA contained in a cell. Alternatively, the nucleic acid may have a sequence complementary to the target sequence. In a case where the component is protein, the protein may be, for example, an antibody. The component may be an aptamer or a molecular imprinted polymer.

The molecule capturing sequence part 15 may include two or more kinds of components for capturing a molecule contained in a cell. The molecule capturing sequence part 15 may include both protein and a nucleic acid, and may include, for example, both an antibody and a polyT sequence. Thereby, both a protein and mRNA can be detected simultaneously.

### (2-6) Bioparticle Capturing Part 16

The bioparticle capturing part 16 includes a component for capturing a bioparticle, and particularly includes a component for capturing a cell. The component may be, for example, an antibody, an aptamer, or an oleyl group. The antibody may be, for example, an antibody that binds to a component (in particular, surface antigens) present on the surface of a bioparticle such as a cell or an exosome. The aptamer may be a nucleic acid aptamer or a peptide aptamer. The aptamer can also bind to a component (in particular, surface antigens) present on the surface of a bioparticle such as a cell or an exosome. The oleyl group can bind a bioparticle formed from a lipid bilayer membrane such as a cell or an exosome.

### (2-7) Collection Sequence Part 17

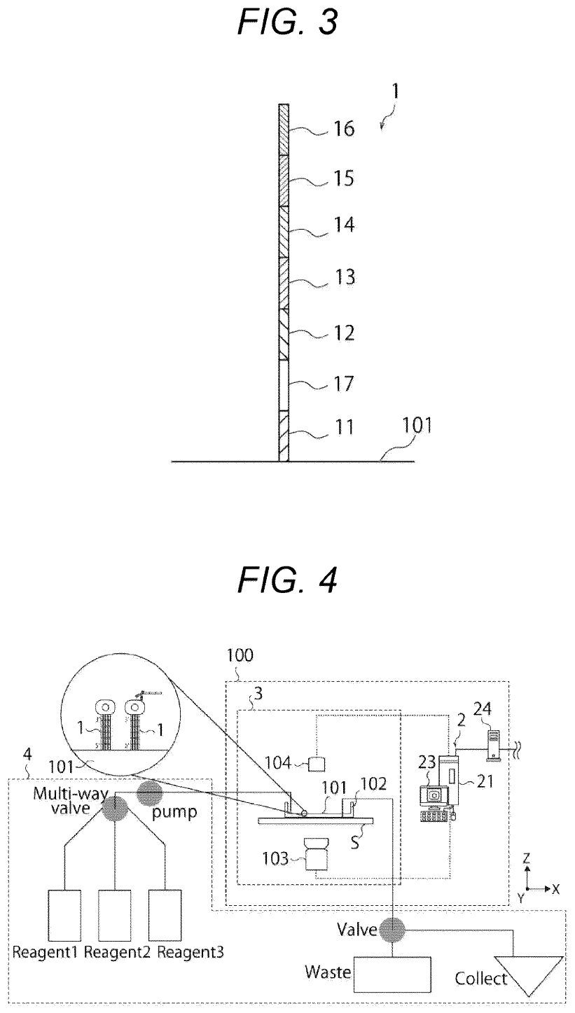

FIG. 3 is a schematic view illustrating one example of an embodiment of the capturing device 1 different from that of FIG. 2. As illustrated in FIG. 2, the capturing device 1 may further include a collection sequence part 17 as illustrated in FIG. 3, in addition to the linker 11, the amplification sequence part 12, the barcode sequence part 13, a unique molecular identifier (UMI) part 14, the molecule capturing sequence part 15, and the bioparticle capturing part 16.

The collection sequence part 17 contains a nucleic acid to be used for collecting the capturing device 1 detached from the bioparticle when the bioparticle is broken. The nucleic acid may be DNA or RNA and is particularly DNA. Note that for the collection, a bead on which a nucleic acid complementary to the nucleic acid is immobilized may be used. With such a bead, the capturing device 1 having the collection sequence part 17 can be efficiently collected. A base sequence of the nucleic acid contained in the collection sequence part 17 may be appropriately set by those skilled in the art.

### (3) Information Processing Device 2

The information processing device 2 includes a processing unit 21. Furthermore, the information processing device 2 may include a storage unit 22, a user interface unit 23, an output unit 24, and the like, as necessary. Note that the respective units of the information processing device 2 may be connected via a network. In addition, a plurality of these units may be provided or may be provided outside such as a cloud and connected via a network.

Hereinafter, each unit of the information processing device 2 will be described in detail.

### (2-1) Processing Unit 21

The processing unit 21 associates morphological information regarding a bioparticle obtained on the basis of the captured image information with information regarding a molecule obtained on the basis of a sequence of the barcode sequence part 13 assigned to the molecule derived from the bioparticle captured by the molecule capturing sequence part 15. A specific method will be described in detail in “(2-5) Association step S5” described later.

Furthermore, the processing unit 21 can analyze all items in the bioparticle analysis system 100 according to the present technology. In addition, a learned model created in “(4-1) Learned model creation step S11” may also be constructed in the processing unit 21. The learned model is a learned model obtained by machine learning and receives input of the morphological information regarding the bioparticle and outputs related molecular information data. This also makes it possible to construct a data set associated with, for example, Morphology, Phenotype, Genotype, and the like, of a cell into which a genetic mutation has been intentionally inserted.

Furthermore, the processing unit 21 can estimate the information regarding the molecule from the morphological information regarding the bioparticle using the constructed learned model. A specific method will be described in detail in “(4-2) Inference step S12” described later.

### (2-2) Storage Unit 22

The storage unit 22 can store all items in the bioparticle analysis system 100 according to the present technology. For example, the morphological information regarding the bioparticle obtained on the basis of the captured image information, the information regarding the molecule obtained on the basis of the sequence of the barcode sequence part 13 assigned to the molecule derived from the bioparticle captured by the molecule capturing sequence part 15, information associating these pieces of information, and the like, are stored. Note that, as the storage unit 22, any matter related to the bioparticle analysis system 100 according to the present technology may be stored using an external storage device, or the like.

Note that the storage unit 22 is not particularly limited in terms of installation location, the number of the storage units 22, and the like, and may be installed on the housing side having the processing unit 21 described above. Furthermore, the storage unit 22 is not an essential component in the information processing device 2 and may be installed outside such as a cloud and connected to the processing unit 21 via a network, or an external storage device may be used.

### (2-3) User Interface Unit 23

The user interface unit 23 is a portion to be operated by the user. The user interface unit 23 presents to the user, all items in the bioparticle analysis system 100 according to the present technology. Furthermore, the user accesses each unit of the information processing device 2 and the imaging device 3 through the user interface unit 23 and controls each unit.

Note that the installation location and the number of the user interface units 23 are not particularly limited, and the user interface unit 23 may be installed on the housing side having the processing unit 21, may be installed in the imaging device 3 to be described later, or may be installed in both of them.

As the user interface unit 23, for example, a display, one or more buttons, a mouse, a keyboard, a touch panel, a portable information terminal, or the like, can be used. Furthermore, the user interface unit 23 is not an essential component in the information processing device 2, and an external display device may be used.

### (2-4) Output Unit 24

The output unit 24 is a portion that receives an instruction from the processing unit 21 and outputs, for example, all items related to the bioparticle analysis system 100 according to the present technology.

Note that the installation location and the number of the output units 24 are not particularly limited, and the output unit 24 may be installed on the housing side having the processing unit 21, may be installed in the imaging device 3 to be described later, or may be installed in both of them. Furthermore, the output unit 24 may receive an instruction from the processing unit 21 and output different content depending on the installation location.

As the output unit 24, a printer, a speaker, a portable information terminal, or the like, can be used. Furthermore, the output unit 24 is not an essential component in the information processing device 2, and an external output device may be used.

### (3) Imaging Device 3

The imaging device 3 images the bioparticle captured on the surface 101. A specific method will be described in detail in “(2-3) Imaging step S3” described later.

### 2. Second Embodiment (Bioparticle Analysis System 100)

### (1) Overall Configuration

An overall configuration of the bioparticle analysis system 100 according to a second embodiment of the present technology will be described with reference to FIG. 4. The bioparticle analysis system 100 according to the present embodiment includes the capturing device 1, the information processing device 2, the imaging device 3, and a fluid control unit 4. Furthermore, other portions may also be provided as needed.

Hereinafter, each portion will be described in detail.

Note that the capturing device 1, the information processing device 2, and the imaging device 3 are similar to those described above, and thus, description thereof will be omitted here.

### (2) Fluid Control Unit 4

As illustrated in FIG. 4, the bioparticle analysis system 100 according to the present technology may be connected to the fluid control unit 4. As a result, seeding of the bioparticle, application of a stimulus to the bioparticle by the drug, staining by a reagent (including a cell surface barcode reagent in which a nucleic acid barcode is bound to the antibody), washing, and cleavage by a reagent (application of the cell barcode) can be automatically performed on the bioparticle analysis system 100. Thereafter, the target molecule is identified through a target molecule analysis step S9 described later.

For example, as illustrated in FIG. 4, the fluid control unit 4 may include a multi-way valve capable of supplying a desired reagent, a desired amount, or the like, from a plurality of reagents (Reagents 1 to 3), a pump, a waste unit, a collect unit, a tube connecting these portions, and the like, but the present technology is not limited thereto.

### 3. Third Embodiment (Bioparticle Analysis System 100)

### (1) Overall Configuration

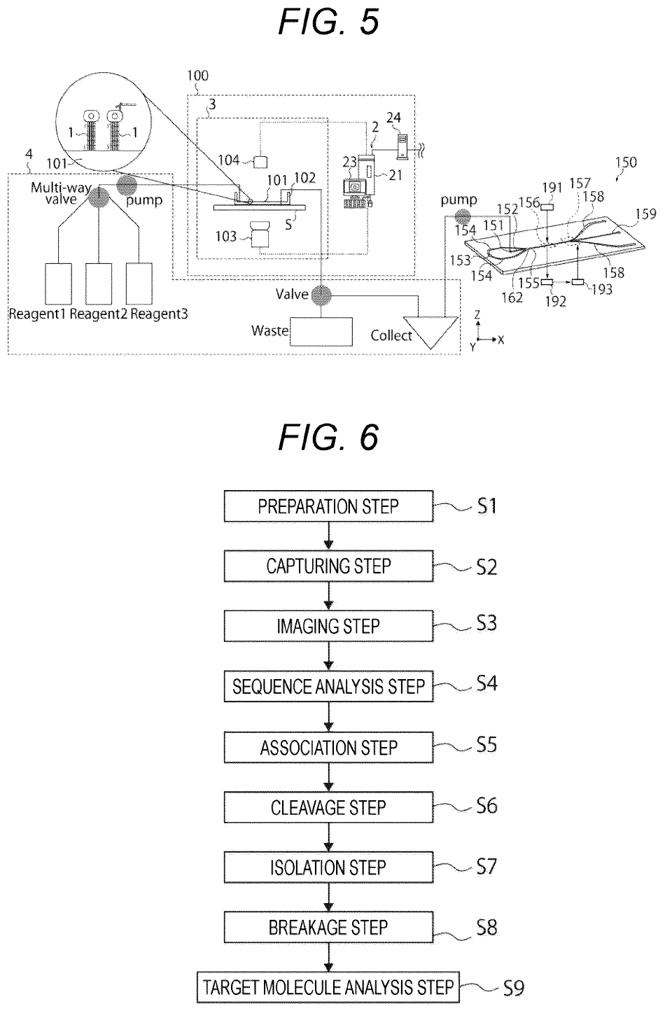

An overall configuration of the bioparticle analysis system 100 according to a third embodiment of the present technology will be described with reference to FIG. 5. The bioparticle analysis system 100 according to the present embodiment includes the capturing device 1, the information processing device 2, the imaging device 3, the fluid control unit 4, and a microchip 150. Furthermore, other portions may also be provided as needed.

Hereinafter, each portion will be described in detail.

Note that the capturing device 1, the information processing device 2, the imaging device 3, and the fluid control unit 4 are similar to those described above, and thus, description thereof will be omitted here.

### (2) Microchip 150

As illustrated in FIG. 5, the microchip 150 may be connected to the bioparticle analysis system 100 according to the present technology. For example, a barcoded cell solution collected by the collect unit of the fluid control unit 4 may be connected to an inlet of the microchip 150 via a collection bag. As a result, an isolation step S7 to a breakage step S8 described later can be continuously performed. Thereafter, the target molecule is identified through a target molecule analysis step S9 described later. The microchip 150 will be described in detail in “(2-7) Isolation step S7” described later.

### 4. Fourth Embodiment (Bioparticle Analysis Method)

### (1) Overall Configuration

The bioparticle analysis method according to the present technology includes a capturing step S2, an imaging step S3, a sequence analysis step S4, and an association step S5. Furthermore, the bioparticle analysis method may include a preparation step S1, a stimulation step S10, a cleavage step S6, an isolation step S7, a breakage step S8, a target molecule analysis step S9, a learned model creation step S11, an inference step S12, and the like.

Hereinafter, each step will be described in detail with reference to the drawings.

### (2) First Flow Example

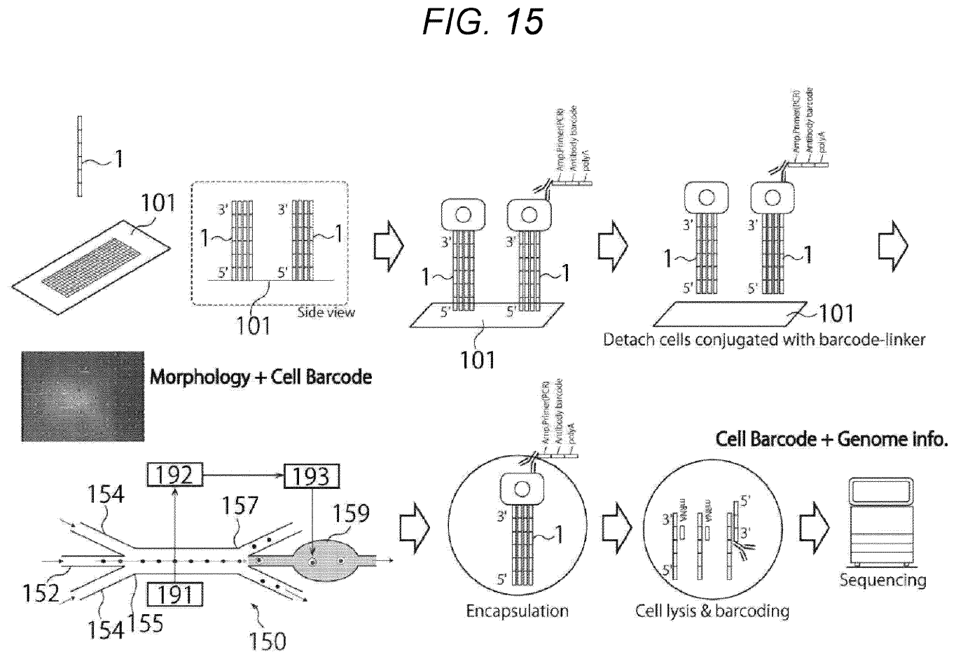

FIG. 6 is a flowchart for explaining a first flow example. An example of flow of the bioparticle analysis method according to the present technology will be described in detail with reference to FIG. 6. Furthermore, FIG. 15 is a schematic view for explaining operation in each step included in the bioparticle analysis method according to the fourth embodiment.

### (2-1) Preparation Step S1

In the preparation step S1, a surface on which the capturing device 1 is immobilized via the linker 11 is prepared. Specifically, for example, an analysis substrate (for example, a glass slide, or the like) 102 having the surface 101 on which a plurality of the capturing devices 1 is immobilized can be prepared. Note that the capturing device 1 has been described above, and thus, the description thereof will be omitted here.

The surface 101 is preferably a surface of a transparent substrate. The entire substrate may be transparent, or only a portion to which the capturing device 1 is immobilized may be transparent. The surface of the substrate is preferably planar for favorable contact with a specimen. The transparent substrate may be, for example, a glass substrate or a resin substrate. The substrate may be, for example, a glass slide. By being transparent, the bioparticle to be cleaved can be easily selected in the cleavage step S6 described later.

The number and density of the capturing devices 1 that bind to the surface 101 can be increased, for example, by increasing a surface area of the surface 101. In addition, a plurality of capturing devices 1 may be connected in series. In this case, a cleavage condition between the substrate 102 and the capturing device 1 is preferably different from a cleavage condition between the two capturing devices 1. If the molecules are also cleaved at the time of cleavage between the substrate 102 and the capturing device 1, the cleaved molecules can be bound to other adjacent bioparticles. However, the cleavage conditions are different from each other, so that it is possible to prevent occurrence of binding.

For example, the linker 11 binding the substrate 102 and the capturing device 1 may be a linker cleavable by light stimulation, and the linker binding the capturing device 1 and the capturing device 1 may be a linker cleavable by chemical stimulation, and vice versa. In addition, the linker binding the substrate 102 and the capturing device 1 may be a linker that is cleaved by chemical stimulation, and the linker binding the capturing device 1 and the capturing device 1 may be a linker cleavable by another chemical stimulation. For example, the former may contain a restriction enzyme identification sequence and the latter may contain other restriction enzyme identification sequences. In addition, for example, the former may contain a disulfide bond, and the latter may contain a restriction enzyme identification sequence. In addition, binding between molecules may be performed with an amino acid, and the binding may be cleaved by a reagent (such as, for example, proteinase K) to be used for cell lysis in the breakage step S8 described later (in particular, at the same time as cell lysis).

### (2-2) Capturing Step S2

In the capturing step S2, the bioparticle (in particular, a cell or a mass of cells) is captured via the bioparticle capturing part 16 on the surface 101 on which the cleavable linker 11, the bioparticle capturing part 16, the molecule capturing sequence part 15, and the barcode sequence part 13 are immobilized via the linker 11. In the capturing step S2, the bioparticle can bind to the bioparticle capturing part 16 in a specific or non-specific manner.

For example, in a case where the bioparticle is a cell or a mass of cells, the cell can be captured by the capturing device 1 by the surface antigen of the cell or the mass of cells binding to the antibody or aptamer contained in the bioparticle capturing part 16. The antibody and the aptamer may be specific or non-specific. Furthermore, in this case, the cell may be captured by the capturing device 1 by the lipid bilayer membrane of the cell binding to the oleyl group contained in the bioparticle capturing part 16. Alternatively, for example, in a case where the bioparticle is an exosome, the bioparticle can be captured by the capturing device 1 by a surface component (that is, the lipid bilayer membrane component) of the bioparticle binding to the oleyl group contained in the bioparticle capturing part 16. Furthermore, in this case, the bioparticle may be captured by the capturing device 1 by a surface component of the exosome binding to the antibody or aptamer contained in the bioparticle capturing part 16.

In addition, the capturing step S2 may include an application step of applying the bioparticle to the surface 101. The application may be performed, for example, by bringing a bioparticle-containing sample (such as, for example, a bioparticle-containing liquid) into contact with the surface 101. For example, the bioparticle-containing sample may be dropped onto the surface 101.

In the capturing step S2, preferably, a plurality of molecules binding to one bioparticle may have the same barcode sequence. Thereby, one barcode sequence can be associated with one bioparticle. Furthermore, preferably, the UMI parts 14 included in the plurality of molecules may have different sequences. Thereby, for example, the number of copies of mRNA can be determined.

Furthermore, in the present technology, an antibody (hereinafter, also referred to as a “nucleic acid-binding antibody”) in which a nucleic acid containing an antibody barcode sequence is bound may be bound to the bioparticle, in particular, a bioparticle surface antigen and bioparticle protein (such as, for example, a transcription factor). In a case where the antibody is bound, membrane permeation treatment may be performed, and examples thereof include treatment with 20 mM Tris HCl, 150 mM NaCl, or 3 mM MgCl₂ (pH7.4) containing 0.01% w/v digitonin. Furthermore, a surfactant such as 1% Tween-20 and 0.1% Nonident P40 substitute may be used. The membrane treatment period depends on the target bioparticle, but the membrane permeation treatment can be performed by treatment for about 1 to 10 minutes. As described above, by selecting an appropriate treatment condition, the membrane is not completely broken, a captured state on the substrate 102 is maintained, and the state in which the barcode is bound can be maintained.

The antibody barcode sequence is a barcode sequence for specifying a nucleic acid-binding antibody. For example, the nucleic acid-binding antibody illustrated in FIG. 10 is bound to the bioparticle instead of or in addition to the fluorochrome-labeled antibody.

The nucleic acid-binding antibody illustrated in FIG. 10 includes an antibody 10 and a nucleic acid binding to the antibody. The nucleic acid includes, for example, a first nucleic acid 201, a second nucleic acid 202, and a third nucleic acid 203 as illustrated in FIG. 10. These nucleic acids may be arranged in the order illustrated in FIG. 10 or may be arranged in other order.

The first nucleic acid 201 may include an amplification primer sequence. As a result of the first nucleic acid 201 including the amplification primer sequence, the barcode sequence part 13 and/or the UMI part 14 included in the capturing device 1 can be added to the second nucleic acid 202 and the third nucleic acid 203 to be described later at the time of amplification. In addition, a sequence for sequencing processing, for example, an adaptor sequence, or the like, can be added.

The second nucleic acid 202 may include an antibody barcode sequence. The antibody barcode sequence may be used to distinguish a nucleic acid-binding antibody binding to one bioparticle from a nucleic acid-binding antibody binding to another bioparticle. For example, the sequence of the antibody barcode sequence may be different for each type of antibody, or the antibody barcode sequence may be different for each type of bioparticle. This results in making it possible to identify the bioparticle to which the nucleic acid-binding antibody has bound after the bioparticle is broken in the breakage step S8 described later.

The third nucleic acid 203 may include a polyA sequence. As a result, after the breakage step S8 described later, the nucleic acid containing the first nucleic acid 201 and the second nucleic acid 202 can be captured by the poly T sequence contained in the molecule capturing sequence part 15 of the capturing device 1 via the third nucleic acid 203. Then, a complex of the nucleic acid and the capturing device 1 is formed by the capturing. For example, by amplifying the complex using the first nucleic acid 201, a nucleic acid to which the antibody barcode sequence of the second nucleic acid 202 is assigned is generated in the capturing device 1. The nucleic acid generated by the amplification has an antibody barcode sequence, and the antibody barcode sequence is different for each type of antibody, for example, associated with the type of antibody, as described above. Thus, information regarding the type and/or number of nucleic acid-binding antibodies is maintained in a product of the amplification in the form of an antibody barcode sequence, and for example, the type and/or number of nucleic acid-binding antibodies associated with the antibody barcode sequence can be identified from the sequence and/or number of nucleic acids having the antibody barcode sequence. This enables the type and number of nucleic acid-binding antibodies binding to the bioparticle to be identified. The identification may be performed, for example, in the later-described target molecule analysis step S9. Sequence analysis of the amplification product for the identification can be performed by, for example, NGS.

The capturing step S2 may include an incubation step for binding the bioparticle and the bioparticle capturing part 16. An incubation condition such as an incubation period and a temperature may be determined according to the type of the bioparticle capturing part 16 to be used.

In addition, after the capturing step S2 is executed, a removal step of removing the bioparticle that has not bound to the capturing device 1 may be performed. In addition, after the capturing step S2 is executed, a removal step of removing unnecessary substances in the cleavage step S6 described later, such as an antibody that has not bound to the bioparticle, may be performed. The removal step may include washing the surface 101 with a liquid such as, for example, a buffer.

### (2-3) Imaging Step S3

In the imaging step S3, the bioparticle captured on the surface by the capturing device 1 is imaged. The imaging is performed on a stage S in a state where the bioparticle is captured on the surface 101. In addition, it is preferable that the imaging is performed with a resolution with which individual bioparticles can be identified. An imaging element 103 may be, for example, a CCD or a COMS. A light source 104 emits light when the captured bioparticle is imaged by the imaging element 103. As the light source 104, for example, a light emitting diode (LED), or the like, that emits light of a specific wavelength is used.

The imaging may be bright-field (including a phase difference) or dark-field imaging, and both bright-field imaging and dark-field imaging may be performed. The imaging may be performed once or a plurality of times, for example, may be performed once or a plurality of times for a partial region selected by a user or a control unit (not illustrated) or may be performed once or a plurality of times so as to cover the entire region or part thereof.

The imaging by the imaging element 103 can be controlled by a control unit (not illustrated) connected to the imaging element. The control unit may be constituted with, for example, a hard disk, a CPU, a memory, or the like, and its function can be implemented by a general-purpose computer, an information processing device, or the like. Furthermore, the control unit may be provided in the above-described imaging element. The imaging element including the control unit may be constituted as, for example, a one-chip semiconductor device having a laminate structure in which a plurality of dies (for example, two or three dies) is laminated. In this configuration, one of the dies includes a plurality of pixels arranged in a two-dimensional manner. Components (such as, for example, a CPU and a memory) for implementing the function of the control unit may be mounted on the remaining dies. Examples of the imaging element including such a control unit include the imaging element disclosed in wo 2018/051809 A, and the like. Use of the imaging element including the control unit as the imaging element enables various types of processing without outputting sample image data to the outside of the imaging element, which results in achieving higher speed of information processing.

The imaging element can transmit captured image information obtained by imaging to the control unit. The control unit receives the captured image information and uses the image data in subsequent steps.

Furthermore, the captured image information received by the control unit may be stored in, for example, a storage unit connected to the control unit. In this case, the storage unit may be a general-purpose storage device. The control unit can acquire the captured image information from the storage unit in a case where the subsequent steps are performed.

### (2-4) Sequence Analysis Step S4

In the sequence analysis step S4, the sequence of the barcode sequence part 13 attached to the molecule (target molecule) derived from the bioparticle captured by the molecule capturing sequence part 15 is analyzed. The sequence analysis step S4 may be performed before the cleavage step S6 described later and may be performed, for example, after the preparation step S1 and before the capturing step S2.

The sequence of the barcode sequence part 13 is analyzed, for example, by reading the barcode sequence of the barcode sequence part 13. The reading can be performed by, for example, a method such as sequencing by synthesis, sequencing by ligation, or sequencing by hybridization.

### (2-5) Association Step S5

In the association step S5, the morphological information on the bioparticle obtained on the basis of the captured image information obtained in the imaging step S3 is associated with the information on the molecule obtained on the basis of the sequence of the barcode sequence part 13 obtained in the sequence analysis step S4.

The association is performed by the information processing device 2 described above, for example, and may be performed via position information (such as, for example, XY coordinates) associated in advance with the barcode sequence part 13. As a result, the barcode sequence at the location where the bioparticle is captured is associated with the captured image information. In a case where an ID number is assigned to each barcode sequence part 13, the captured image of the bioparticle, the feature amount extracted from the captured image, and the ID number may be associated with each other. As a result, the captured image and the feature amount extracted from the captured image can be associated with the barcode sequence part 13 via the ID number.

### (2-6) Cleavage Step S6

In the cleavage step S6, the linker 11 is cleaved. By cleavage of the linker 11, the bioparticle to which the molecule binds is detached from the surface 101. For example, when the linker 11 of the capturing device 1 is cleaved, the capturing device 1 is detached from the surface 101, and accordingly, the bioparticle is also detached from the surface 101.

In the cleavage step S6, for example, the linker 11 can be cleaved by stimulation such as chemical stimulation or light stimulation. The light stimulation is suitable because it can selectively stimulate a specific narrow range. A stimulus can be applied by a stimulus application device. Drive of the stimulus application device may be controlled by, for example, an information processing device such as a general-purpose computer. For example, the information processing device may drive the stimulus application device to cause the stimulus application device to selectively apply a stimulus to a position of the bioparticle to be detached.

As the stimulus application device that applies a light stimulus to a selective position of the bioparticle, for example, a light irradiation device is used. Specific examples thereof include a digital micromirror device (DMD) and a liquid crystal display device. A selected position on the surface 101 can be irradiated with light by the micromirror constituting the DMD. The liquid crystal display device may be, for example, a reflective liquid crystal display, and examples thereof include SXRD (manufactured by Sony Corporation). By controlling a liquid crystal of the liquid crystal display device, selective positions of the surface 101 may be irradiated with light. In addition, a liquid crystal shutter or a spatial light modulator may be used to apply a light stimulus to the selective position of the bioparticle. These can also provide a light stimulus to the selective position. A wavelength of the light radiated by the light irradiation device may be appropriately selected by those skilled in the art according to the type of the linker 11 included in the capturing device 1.

The chemical stimulation may be performed by bringing a reagent for cleaving the linker 11 into contact with the surface 101. The reagent may be appropriately selected by those skilled in the art according to the type of the linker 11. For example, in a case where the linker 11 contains a disulfide bond, the reagent may be a reducing agent capable of cleaving the bond, and examples thereof include Tris(2-carboxyethyl) phosphine (TCEP), Dithiothreitol (DTT), and 2-Mercaptoethanol. In addition, for example, in a case where the linker 11 is a nucleic acid containing a restriction enzyme identification sequence, the reagent may be a restriction enzyme corresponding to each restriction enzyme identification sequence.

In the cleavage step S6, at least one bioparticle detached by cleavage may be collected, for example, in a liquid such as a buffer. The liquid may be, for example, a hydrophilic liquid. The bioparticle-containing liquid obtained by the collection can be used in the isolation step S7 described later. In order to collect the detached bioparticle, a fluid force by flowing the liquid such as a buffer may be used, the bioparticle may be floated in the liquid by vibration, or the bioparticle may be floated in the liquid using gravity, or the like. The vibration may be, for example, vibration of the analysis substrate 102 or vibration of the liquid containing the bioparticle. In addition, the analysis substrate 102 may be moved such that the surface 101 faces the direction of gravity in order to float the bioparticle in the liquid by the gravity.

### (2-7) Isolation Step S7

In the isolation step S7, the bioparticle detached from the surface 101 in the cleavage step S6 is isolated in the microspace. By the isolation, the capturing device 1 can be bound to, for example, a target substance contained in the bioparticle. As a result, for example, the barcode sequence part 13 included in the capturing device 1 can be associated with a molecule (that is, the target molecule) derived from the bioparticle. The target molecule can be analyzed using the information of the barcode sequence part 13, and in particular, single cell analysis can be performed.

The microspace may be, for example, a space in an emulsion particle or a space in a well. Preferably, in the isolation step S7, one bioparticle (in particular, one bioparticle to which at least one capturing device 1 binds) is isolated in one emulsion particle or one well.

In an embodiment of the present technology, the isolation step S7 can include a discrimination step (not illustrated) of discriminating whether or not to isolate the bioparticle in the microspace, and a particle isolation step (not illustrated) of isolating the bioparticle discriminated to be isolated in the discrimination step in the microspace. This makes it possible to isolate only the target bioparticle. Thus, for example, bioparticles other than the target bioparticle can be excluded from the target in the target molecule analysis step S9 described later.

The discrimination may be performed, for example, on the basis of light (for example, scattered light and/or autofluorescence) generated from the bioparticle, light generated from a substance binding to the bioparticle, a morphology image of the bioparticle, or the like. The substance binding to the bioparticle may be, for example, the capturing device 1 or an antibody (in particular, fluorochrome-labeled antibodies) binding to the bioparticle. The scattered light generated from the bioparticle may be, for example, forward scattered light and/or side scattered light. Doublet detection can be performed from a height of a signal acquired by the scattered light detection, an area value, and the like. Furthermore, single cell determination can also be performed by the morphological image of the bioparticle. Whether or not the bioparticle is a dead cell can be discriminated from scattered light and/or a morphological image, fluorescence after staining with a dead cell staining reagent, and the like, whereby the dead cell can be removed. Furthermore, in the present technology, the discrimination step may be performed immediately before the isolation step S7, whereby only the single cell to which the barcode is assigned can be reliably isolated.

Furthermore, in another embodiment of the present technology, only the particle isolation step may be executed without executing the discrimination step. This can reduce the number of steps in the bioparticle analysis method according to the present technology.

In still another embodiment of the present technology, the discrimination step may be performed in the cleavage step S6 described above instead of being performed in the isolation step S7. In this case, the bioparticle or the bioparticle population selected as a result of the discrimination in these steps is subjected to the particle isolation step. In this case, for example, a device such as a cell sorter does not have to be used.

### (2-7-1) Discrimination Step

In the discrimination step, it is discriminated whether to isolate the detached bioparticle in a microspace. The discrimination may be performed on the basis of light generated from the bioparticle or light generated from a substance binding the bioparticle. In this case, the discrimination step may include, for example, an irradiation step of irradiating the bioparticle with light, and a detection step of detecting light generated by the irradiation.

The irradiation step may be executed by, for example, a light irradiation unit that irradiates the bioparticle with light. The light irradiation unit may include, for example, a light source that emits light. In addition, the light irradiation unit may include an objective lens that condenses light on the bioparticle. The light source may be appropriately selected by those skilled in the art according to the purpose of analysis. The light irradiation unit may include other optical elements in addition to the light source and the objective lens.

The detection step may be executed by, for example, a detection unit that detects light generated from the bioparticle or the substance binding to the bioparticle. The detection unit detects, for example, light (for example, scattered light and/or fluorescence) generated from the bioparticle or the substance binding to the bioparticle by light irradiation by the light irradiation unit. The detection unit may include, for example, a condenser lens that condenses light generated from the bioparticle, and a detector. The detection unit may include other optical elements as necessary in addition to the condenser lens and the detector. For example, the detection unit may further include a spectroscopic unit. The spectroscopic unit can detect, for example, light having a wavelength that should be detected separately from light having other wavelengths. The detection unit may convert the detected light into an analog electric signal by photoelectric conversion and further convert the analog electric signal into a digital electric signal by AD conversion.

In addition, determination processing as to whether or not to discriminate the bioparticle on the basis of the light detected in the detection step may be executed by a determination unit (not illustrated). The processing by the determination unit may be implemented by, for example, an information processing device such as a general-purpose computer, in particular, a processing unit included in the information processing device according to the present technology.

### (2-7-2) Particle Isolation Step

The particle isolation step isolates the bioparticle in a microspace. In the present specification, the “microspace” may mean a space having a dimension capable of accommodating one bioparticle to be analyzed. The dimension may be appropriately determined by those skilled in the art according to factors such as a size of the bioparticle, for example. The microspace may also have a dimension capable of accommodating two or more bioparticles to be analyzed. This case may include, in addition to a case where one bioparticle is accommodated in one microspace, a case where two or more bioparticles are accommodated. The bioparticles in the microspace in which two or more bioparticles are accommodated may be excluded from the target of breakage in the breakage step S8 to be described later or may be excluded from the target of analysis in the target molecule analysis step S9 to be described later.

In the breakage step S8 described later, for example, a complex of a target molecule and a target molecule can be generated. The plurality of microspaces to be used in the present technology is preferably separated from each other so that the complex generated in one microspace does not shift to another microspace. Examples of the microspace separated in this manner include a space in a well and a space in an emulsion particle. In other words, in a preferred embodiment of the present technology, the microspace may be a space in a well or a space in an emulsion particle.

### (2-7-2-1) In Case of Space in Well

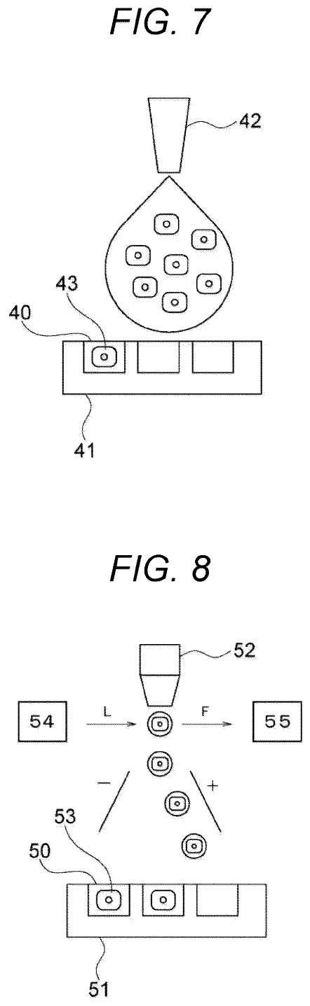

FIG. 7 is a schematic view of an example of a well to be used to execute the particle isolation step. As illustrated in FIG. 7, for example, a plurality of wells 40 each having a dimension capable of accommodating one bioparticle may be formed on the surface of the substrate 41. By applying the bioparticle-containing liquid obtained in the above-described cleavage step S6 to the surface of the substrate 41 from, for example, an arbitrary nozzle 42, the bioparticle 43 is isolated in the space in the well 40 as illustrated in FIG. 7. In this manner, one bioparticles may be put in a space of one well and isolated in the microspace.

In addition, as illustrated in FIG. 7, in a case where a liquid containing a plurality of bioparticles is applied to a substrate on which a well is formed, the particle isolation step may be executed without executing the above-described discrimination step. On the other hand, in a case where the discrimination step is performed, for example, a device capable of putting one bioparticle in one well such as a cell sorter or a single cell dispenser may be used. Also for the device, a substrate (such as, for example, a plate) on which a plurality of wells is formed can be used to isolate the bioparticle. As the device, a commercially available device may be used. The device may include, for example, a light irradiation unit that radiates light, a detection unit that detects light, a discrimination unit that discriminates whether or not to put the bioparticle into the well on the basis of the detected light, a distribution unit that distributes the bioparticle determined to be put into the well to the well, and the like. The distribution unit may include a microfluidic chip having a nozzle that forms a droplet containing a bioparticle.

The device operates a position of the microfluidic chip according to the discrimination result by the discrimination unit to put one bioparticle-containing droplet into a predetermined well. Alternatively, the device controls, according to the discrimination result by the discrimination unit, a traveling direction of the bioparticle-containing droplet discharged from the nozzle using a charge applied to the droplet. One bioparticle-containing droplet is put into a predetermined well by the control. In this manner, one bioparticle is distributed in one well. For example, as illustrated in FIG. 8, the bioparticle-containing droplet is discharged from the nozzle 52 provided in the microfluidic chip of the device. The bioparticle contained in the droplet is irradiated with light (for example, laser light L) by the light irradiation unit 54, and then, a detection step is executed by the detection unit 55, and light (fluorescence F) is detected. Then, the discrimination unit executes a determination step on the basis of the detected light. Then, the distribution unit controls, according to the determination result, the traveling direction of the droplet using the charge applied to the droplet. By the control, a droplet containing the target bioparticle is collected in a predetermined well. As a result, one bioparticle is distributed in one well.

By executing the discrimination by the discrimination unit, for example, it is possible to identify a cell population to which the bioparticle belongs, identify the bioparticle to which the barcode is assigned, and identify the droplet containing the singlet bioparticle according to the detection signal. This enables collection of only a droplet containing the target bioparticle. As a result, it is not necessary to exclude data in the target molecule analysis step S9 described later, which improves analysis efficiency.

### (2-7-2-2) In Case of Space in Emulsion Particle

The emulsion particle may be produced using, for example, a microfluidic channel. The device includes, for example, a channel through which a first liquid forming an emulsion dispersoid flows and a channel through which a second liquid forming a dispersion medium flows. In this case, the first liquid may contain a bioparticle. The device may further include a region where the two liquids come into contact with each other to form an emulsion.

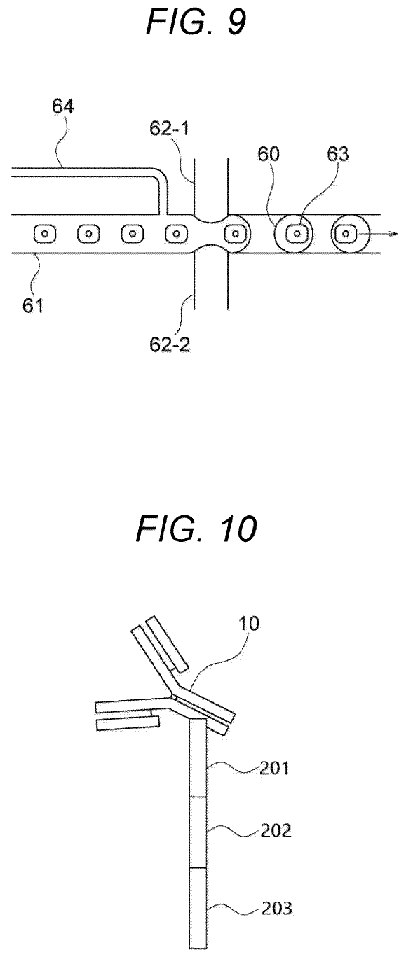

The microchannel illustrated in FIG. 9 includes a channel 61 through which the first liquid containing bioparticles flows and channels 62-1 and 62-2 through which the second liquid flows, and the first liquid forms an emulsion particle (dispersoid) and the second liquid forms a dispersion medium of an emulsion. The channel 61 and the channels 62-1 and 62-2 merge, and an emulsion particle is formed at this junction. Then, the bioparticle 63 is isolated inside the emulsion particle. For example, a size of the emulsion particle can be controlled by controlling the flow velocity in these channels. In order to form an emulsion, the first liquid and the second liquid are immiscible with each other. For example, the first liquid may be a hydrophilic liquid, and the second liquid may be a hydrophobic liquid, or vice versa. The microchannel illustrated in FIG. 9 may include a channel 64 for introducing the bioparticle-breaking substance into the emulsion particle. By configuring the microchannel such that the channel 64 merges with the channel 61 immediately before the junction, it is possible to prevent the bioparticle from being broken by the bioparticle-breaking substance before the emulsion particle is formed.

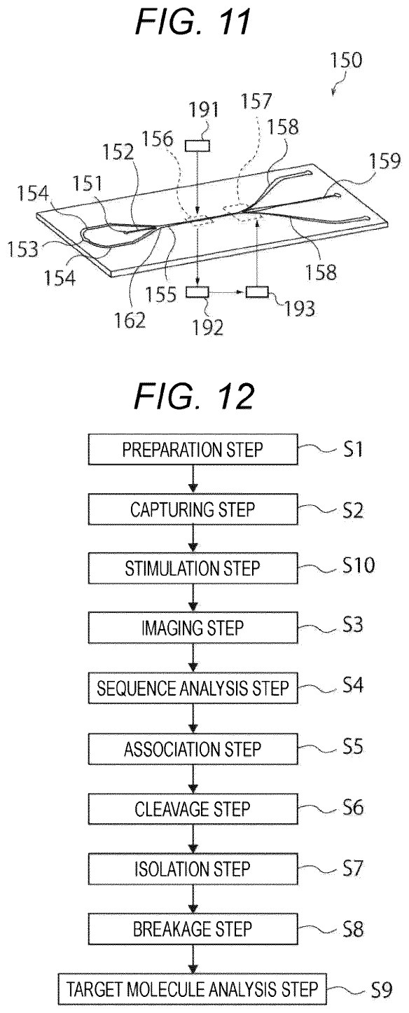

Next, an example of a device for more efficiently forming an emulsion containing an emulsion particle containing one bioparticle will be described with reference to FIG. 11. By the emulsion forming device, one bioparticle can be isolated in one emulsion particle with extremely high probability, and the number of empty emulsion particles can be reduced. Furthermore, a probability of isolating one bioparticle and one barcode sequence in one emulsion particle is also increased by the emulsion forming device.

FIG. 11 is a view schematically illustrating one example of an embodiment of a microchip to be used for forming an emulsion particle in the device. The microchip 150 illustrated in FIG. 11 is provided with a sample liquid inlet 151 and a sheath liquid inlet 153. Furthermore, from these inlets, a sample liquid containing a bioparticle and a sheath liquid not containing a bioparticle are introduced into a sample liquid channel 152 and a sheath liquid channel 154, respectively. The microchip 150 has a channel structure in which the sample liquid channel 152 through which the sample liquid flows merges with the sheath liquid channel 154 through which the sheath liquid flows at a merging portion 162 to become a main channel 155. The sample liquid merges with the sheath liquid at the merging portion 162 to form, for example, laminar flow in which the sample liquid is enclosed by the sheath liquid. The laminar flow flows through the main channel 155 toward a particle sorting unit 157. Preferably, the bioparticles flow in a line in the main channel 155. In the main channel 155, the bioparticles are irradiated with light in a detection region 156.

The detection unit 192 detects light generated by the light irradiation. According to the characteristics of the light detected by the detection unit 192, a determination unit included in the control unit 193 determines whether or not the bioparticle is a particle to be collected. In the particle sorting unit 157, only in a case where the particle to be collected flows, flow entering a collection channel 159 from the main channel 155 is formed, and the particle to be collected is collected into the collection channel 159. On the other hand, microparticles that are not the particle to be collected flow to a waste channel 158.

In addition, the microchip 150 can constitute part of a bioparticle sorting device including the light irradiation unit 191, the detection unit 192, and the control unit 193 in addition to the microchip 150. The control unit 193 includes a signal processing unit, a determination unit, and a sorting control unit. In other words, the bioparticle sorting device can be used as the above-described emulsion forming device.

In order to form an emulsion containing an emulsion particle containing one target bioparticle, for example, in the microchip 150, a flow passing step of flowing the first liquid containing the bioparticle to the main channel 155, a discrimination step of determining whether or not the bioparticle flowing through the main channel 155 is a particle to be collected, and a collection step of collecting the particle to be collected into the collection channel 159 can be executed. Here, the discrimination step corresponds to the discrimination step described in (2-7-1) described above. The collection step corresponds to the particle isolation step described in (2-7-2) described above.

### (2-8) Breakage Step S8

In the breakage step S8, the bioparticle is broken in the microspace. With the breakage, for example, the capturing device 1 that has bound to the bioparticle via the bioparticle capturing part 16 may be dissociated from the bioparticle. Note that, among the components of the broken bioparticle, the component that has bound to the bioparticle capturing part 16 may bind to the capturing device 1 via the bioparticle capturing part 16 even after the breakage.

In the breakage step S8, the target molecule constituting or binding to the bioparticle is captured by the molecule capturing sequence part 15 included in the capturing device 1. As a result, a complex of the capturing device 1 and the target molecule is formed, and the target molecule can be associated with the barcode sequence part 13 included in the capturing device 1 in the target molecule analysis step S9 described later. In other words, the complex thus formed is analyzed in the target molecule analysis step S9 described later.

The breakage step S8 is preferably executed while a state where the bioparticle is isolated into the microspace is maintained. As a result, the complex of the capturing device 1 and the target molecule is efficiently formed. In addition, it is possible to prevent the target molecule from binding to the molecule capturing sequence part 15 existing outside the microspace. In a case where the microspace means a space within the emulsion particle, maintaining the isolation may mean maintaining the emulsion particle, particularly meaning that the emulsion particle is not broken. In a case where the microspace means a space in a well, maintaining the isolation state may mean that a component in the well remains in the well, and may further mean that a component in another well does not enter the well.

In addition, in a case where the nucleic acid-binding antibody binds to the bioparticle in the capturing step S2 described above, the nucleic acid-binding antibody is dissociated from the bioparticle in the breakage step S8. Then, the nucleic acid-binding antibody binds to the target molecule, and a complex of the nucleic acid-binding antibody and the target molecule can be formed. For example, the polyA sequence constituting the first nucleic acid 201 can bind to the bioparticle mRNA that is a target substance. The second nucleic acid 202 containing the antibody barcode sequence binds to the first nucleic acid 201, and thus, the target molecule can be associated with the antibody barcode sequence. The complex thus formed is analyzed in the target molecule analysis step S9 described later.

Further, the breakage step S8 can be executed by chemically or physically breaking the bioparticle.

For chemical breakage of the bioparticle, a bioparticle breaking substance and the bioparticle may be brought into contact with each other in the microspace. The bioparticle breaking substance may be appropriately selected by those skilled in the art according to the type of the bioparticle. In a case where the bioparticle is a cell or an exosome, for example, a lipid bilayer membrane breaking component may be used as the bioparticle breaking substance, and specific examples thereof include a surfactant, an alkali component, and an enzyme.

In a case where the microspace is a space in the well, for example, the breakage is performed by adding the bioparticle breaking substance to each well. Each well is isolated from each other, and thus, the components in the well are maintained in the well even when the breakage is performed. Furthermore, in a case where the microspace is a space in an emulsion particle, for example, the bioparticle breaking substance may be introduced into the emulsion particle simultaneously with formation of the emulsion particle. Then, after the emulsion particle is formed, the bioparticle may be broken by the bioparticle breaking substance.

For physical breakage of the bioparticle, a physical stimulus that breaks the bioparticle may be applied to the bioparticle. Examples of the treatment for applying the physical stimulus to the bioparticle include optical treatment, thermal treatment, electrical treatment, acoustic treatment, freeze-thaw treatment, and mechanical treatment. By these kinds of treatment, a cell or an exosome can be broken. The physical breakage of the bioparticle by these kinds of treatment can be applied to both a case where the microspace is a space in a well and a case where the microspace is a space in an emulsion particle. In a case where the microspace is a space in the emulsion particle, optical treatment, thermal treatment, electrical treatment, and freeze-thaw treatment are particularly suitable.

In the breakage step S8, the collection sequence part 17 included in the capturing device 1 may be used. A target molecule may bind to the capturing device 1, and the target molecule can be efficiently collected by using the collection sequence part 17. In other words, the breakage step S8 may include a step of collecting the capturing device 1 (in particular, the target molecule binding to the capturing device 1) using the collection sequence part 17.

### (2-9) Target Molecule Analysis Step S9

In the target molecule analysis step S9, the bioparticle is analyzed. In particular, in the target molecule analysis step S9, the target molecule is analyzed. A method of the analysis may be appropriately determined by those skilled in the art according to the type of the target molecule and the purpose of the analysis.

In the present technology, in the target molecule analysis step S9, the sequence of the barcode sequence part 13 is associated with the target molecule. Specifically, in the association step S5 described above, the morphological information regarding the bioparticle obtained on the basis of the captured image information and the analysis result obtained by the sequence of the barcode sequence part 13 assigned to the target molecule are linked via the sequence. This makes it possible to associate Morphology, Phenotype, Genotype, and the like, and in particular, in a case where a cell modified using a genetic modification technology such as genome editing is prepared, it is also possible to construct a data set in which Morphology, Phenotype, Genotype, and the like, of a cell into which a genetic mutation is intentionally inserted are associated.

In the isolation step S7 described above, one bioparticle is isolated in one microspace, and all of the plurality of capturing devices 1 capturing the bioparticles have the same sequence of the barcode sequence part 13. Thus, all of the analysis results associated with the sequence of one barcode sequence part 13 by executing the association are derived from one bioparticle, whereby the morphological information for analysis of the one bioparticle and the analysis results can be coupled via the barcode sequence.

In the target molecule analysis step S9, the capturing device 1 including the sequence of the barcode sequence part 13 is bound to the target molecule in the above-described breakage step S8, and thus, the analysis result can be associated with each bioparticle on the basis of the sequence even in a case where different bioparticles existing in a plurality of microspaces are collectively analyzed.

For example, in a case where the microspace is a space in a well, bioparticle breaking products in the respective wells may be separately analyzed, or the bioparticle breaking products of a plurality of wells may be collected as one sample, and the one sample may be collectively analyzed. In the former case, it is easy to associate the bioparticle with the analysis result. Also in the latter case, the target molecule in each bioparticle breaking product forms a complex with the capturing device 1 including the sequence of the barcode sequence part 13 or the nucleic acid binding antibody including the sequence of the antibody barcode, so that each bioparticle can be associated with the analysis result thereof.

Furthermore, in a case where the microspace is a space in an emulsion particle, a plurality of emulsion particles may be collectively analyzed, and for example, the entire obtained emulsion may be collectively analyzed. The target molecule in each bioparticle breaking product forms a complex with the capturing device 1 including the barcode sequence or the nucleic acid-binding antibody including the antibody barcode sequence, so that each bioparticle can be associated with the analysis result thereof. This can improve analysis efficiency.

In a case where the bioparticle is analyzed in the target molecule analysis step S9, for example, the analysis may be performed on the complex of the capturing device 1 and the target molecule formed in the breakage step S8 and/or may be performed on the complex of the nucleic acid-binding antibody and the target molecule. The capturing device 1 and the nucleic acid-binding antibody each include the sequence of the barcode sequence part 13 and the sequence of the antibody barcode, so that it is possible to identify the bioparticle from which the target molecule is derived on the basis of these sequences.

In a case where the target molecule has a base sequence, specifically, for example, in a case where the target molecule is RNA (in particular, mRNA) or DNA, sequencing processing may be performed on the base sequence of the target substance in the target molecule analysis step S9. The sequencing processing may be executed by, for example, a next-generation sequencer.

The analysis in the target molecule analysis step S9 may be executed using, for example, the amplification sequence part 12 included in the capturing device 1. In other words, the target molecule analysis step S9 includes a nucleic acid amplification step using the amplification sequence part 12. This can, for example, amplify the nucleic acid (in particular, mRNA) binding to the capturing device 1. Then, information regarding the nucleic acid can be acquired by performing sequencing processing on the sequence of the nucleic acid. In addition, with the amplification, the sequence of the barcode sequence part 13 can also be amplified. As a result, the information regarding the nucleic acid can be associated with the sequence of the barcode sequence part 13 included in the capturing device 1, and further, can be associated with the bioparticle.

The target molecule analysis step S9 may be performed using an analysis device. The analysis device may be, for example, a device that performs sequencing processing on the complex. The sequencing processing is performed, for example, in a case where the target molecule is a nucleic acid, particularly DNA or RNA, more particularly mRNA. The sequencing processing may be performed by a sequencer or may be performed by a next-generation sequencer or a sequencer employing a Sanger method. In order to perform comprehensive analysis of a plurality of bioparticles (in particular, a cell population) at a higher speed, the sequencing processing may be performed by a next-generation sequencer.

In the target molecule analysis step S9, the components can be analyzed for each bioparticle on the basis of the sequencing processing result. For example, in the target molecule analysis step S9, the sequence of mRNA contained for each bioparticle and/or the number of copies of each mRNA can be determined. In addition, in the target molecule analysis step S9, a type and/or the number of antigens and a type and/or the number of transcription factors can be determined for each bioparticle. Such analysis of the components for each bioparticle can be performed on the basis of the sequence of the barcode sequence in the sequence determined by the sequencing processing. For example, a sequence including a sequence of the same barcode sequence is selected from a plurality of sequences of barcode sequences determined by the sequencing processing. A sequence including the same barcode sequence is based on a target molecule incorporated into one cell. Thus, analyzing the components for each sequence of the barcode sequence means analyzing the components for each bioparticle.

### (3) Second Flow Example

FIG. 12 is a flowchart for explaining a second flow example. An example of flow of the bioparticle analysis method according to the present technology will be described in detail with reference to FIG. 12. Note that the preparation step S1, the capturing step S2, the imaging step S3, the sequence analysis step S4, the association step S5, the cleavage step S6, the isolation step S7, the breakage step S8, and the target molecule analysis step S9 are similar to those described above, and thus, the description thereof will be omitted here.

### (3-1) Stimulation Step S10

The second flow example further includes a stimulation step S10 after the capturing step S2. In the stimulation step S10, a stimulus by a drug is applied to the bioparticle. As a result, it is possible to perform observation over time by application of a stimulus, and it is possible to obtain morphological information including a feature amount by observing a drug response, drug resistance, and the like. As a result, as an application to drug discovery screening, a high-throughput and low-cost method can be obtained. After the stimulation step S10, as indicated in FIG. 12, the processing proceeds to an imaging step S3.

The stimulus is appropriately selected by those skilled in the art according to the captured bioparticle. For example, in a case of a T cell, examples of the stimulus include an antigenic stimulus that can be recognized by a T cell receptor, a tetramer or pentamer on which an antigen is immobilized, an anti-CD3 antibody that promotes proliferation, an anti-CD3/CD28 antibody, and the like. In addition, examples can also include a cytokine such as IL-2, IL-7, IL-15, and IL-22. In a case of a B cell, the stimulus may be an antigen stimulus that can be recognized by a B cell receptor, and examples of the stimulus include a tetramer and a pentamer on which an antigen is immobilized. In a case of a patient-derived cancer cell or a cancer cell line, a drug approved as an anticancer agent can be selected. Examples of the anticancer agent include a cytotoxic anticancer agent, a molecular targeting agent (such as, for example, a small molecule compound (such as, for example, a tyrosine kinase inhibitor, a multikinase inhibitor, and a mTOR inhibitor), an antibody drug (such as, for example, an anti-HER2 antibody drug, and an anti-epidermal growth factor receptor antibody), and a nucleic acid drug), an endocrine therapeutic agent, and the like.

In addition, by adding a drug, for example, it is also possible to seed and capture a cell, or the like, with a low density and then proliferate the cell. It is therefore also possible to integrate the morphological information including the feature amount and the molecular information of the cell proliferated by receiving stimulation response.

### (4) Third Flow Example

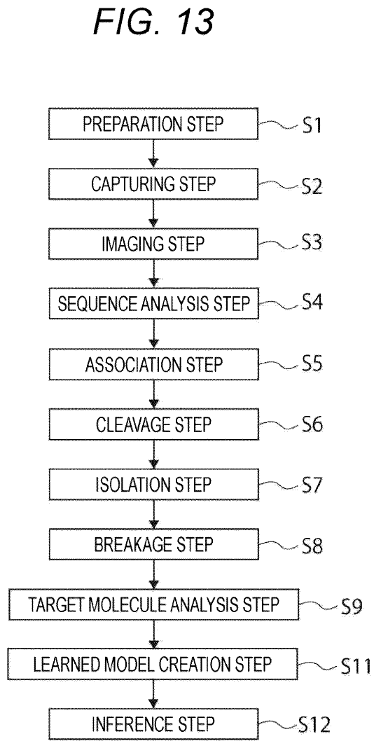

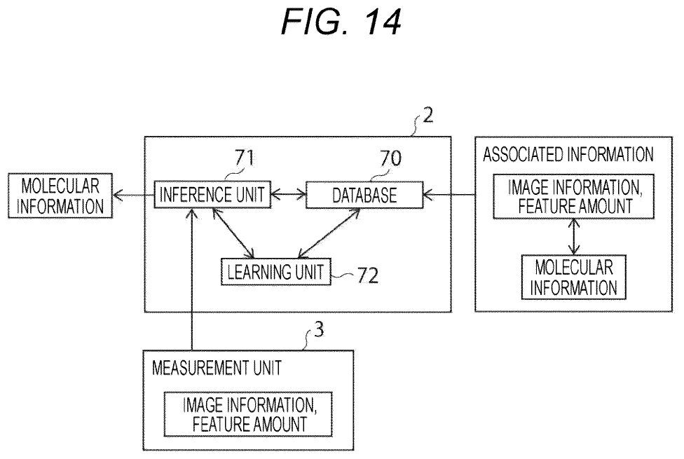

FIG. 13 is a flowchart for explaining a third flow example. One example of flow of the bioparticle analysis method according to the present technology will be described in detail with reference to FIG. 14. Note that the preparation step S1, the capturing step S2, the imaging step S3, the sequence analysis step S4, the association step S5, the cleavage step S6, the isolation step S7, the breakage step S8, and the target molecule analysis step S9 are similar to those described above, and thus, the description thereof will be omitted here.

### (4-1) Learned Model Creation Step S11

The third flow example further includes a learned model creation step S11 after the target molecule analysis step S9. In the stimulation step S10, a learned model is created using the morphological information regarding the bioparticle and the information regarding the target molecule.

Specifically, a data set is constructed using data in which the morphological information regarding the bioparticle obtained on the basis of the captured image information is associated with the analysis result obtained by the barcode sequence assigned to the target molecule, the morphological information being linked to the analysis result in the target molecule analysis step S9 described above. Then, for example, a plurality of data sets are stored in the information processing device 2 to create a database.

### (4-2) Inference Step S12

FIG. 14 is a conceptual diagram illustrating an inference step S12. A database 70 created in the above-described learned model creation step S11 is linked to, for example, an inference unit 71 and a learning unit 72 and estimates the information regarding the molecule on the basis of the captured image information obtained via the measurement unit (in particular, the imaging device 3 described above) illustrated in FIG. 14 and the morphological information obtained on the basis of the captured image information. For example, an explanatory variable is set as a feature amount extracted from the captured image information, a target variable is information regarding the target molecule, and the molecular information derived from the bioparticle can be inferred from the inference unit 71. It is therefore not necessary to perform molecular assay.

In addition, if the information regarding the target molecule can be obtained from a non-stained image, time and cost are also reduced. Furthermore, a cell configuration can be specified in a non-staining manner for a cell to be used for cell therapy, and optimal culture conditions can be presented. In addition, even without antigen information, activated T cell and B cell can be identified, so that application to cell therapy and antibody development can be expected. In other words, the bioparticle analysis method according to the present technology is also useful in applications in which it is desired to avoid staining using a reagent, or the like.

Examples of the molecular information derived from the bioparticle include identification of a cell type (including a subtype), identification of a genetic mutation (such as, for example, whether or not a drug resistance gene exists), and identification of a cell state such as a cell cycle and activity/inactivity (such as whether or not there is antigen-specific reaction of an immune cell, in particular, a T cell). In addition, the learned model (including a prediction model) can be constructed by integrating the morphological information regarding the bioparticle and the information regarding the target molecule at a high throughput and low cost at a single cell level, so that it is possible to save labor and cost for directly measuring the target molecule.

Note that the present technology can also adopt the following configurations.

[1]

A bioparticle analysis system including:
a capturing device that captures a bioparticle via a bioparticle capturing part on a surface on which a cleavable linker, the bioparticle capturing part, a molecule capturing sequence part, and a barcode sequence part are immobilized via the linker; and
an information processing device that associates morphological information regarding the bioparticle obtained on the basis of captured image information with information regarding a molecule obtained on the basis of a sequence of the barcode sequence part assigned to the molecule derived from the bioparticle captured by the molecule capturing sequence part.
   [2]

The bioparticle analysis system according to [1], in which the information processing device extracts a feature amount on the basis of the morphological information regarding the bioparticle.

[3]

The bioparticle analysis system according to [1] or [2], in which the barcode sequence part is associated with position information of the surface in advance.

[4]

The bioparticle analysis system according to [2], in which
an ID number is assigned to the barcode sequence part, and
the information processing device associates the feature amount with the ID number.
   [5]

The bioparticle analysis system according to any one of [1] to [4], in which the bioparticle is stimulated by a drug.

[6]

The bioparticle analysis system according to any one of [1] to [5], in which
the information processing device creates a learned model obtained by machine learning, and
the learned model receives input of the morphological information regarding the bioparticle and outputs related molecular information data.
   [7]

The bioparticle analysis system according to [6], in which the information regarding the molecule is estimated from the morphological information regarding the bioparticle using the learned model.

[8]

The bioparticle analysis system according to any one of [1] to [7], further including an imaging device that images the bioparticle captured on the surface.

[9]

The bioparticle analysis system according to any one of [1] to [8], in which the bioparticle is a cell or a mass of cells.

[10]

An information processing device that associates morphological information regarding a bioparticle obtained on the basis of captured image information with information regarding a molecule obtained on the basis of a barcode sequence part assigned to the molecule derived from the bioparticle.

[11]

A bioparticle analysis method including:
a capturing step of capturing a bioparticle via a bioparticle capturing part on a surface on which a cleavable linker, the bioparticle capturing part, a molecule capturing sequence part, and a barcode sequence part are immobilized via the linker;
an imaging step of imaging the bioparticle captured on the surface;
a sequence analysis step of analyzing a sequence of the barcode sequence part assigned to a molecule derived from the bioparticle captured by the molecule capturing sequence part; and
an association step of associating morphological information regarding the bioparticle obtained on the basis of captured image information obtained in the imaging step with information regarding the molecule obtained on the basis of the sequence of the barcode sequence part obtained in the sequence analysis step.
   [12]

The bioparticle analysis method according to [11], further including a stimulation step of stimulating the bioparticle by a drug.

[13]

The bioparticle analysis method according to [11] or [12], further including a learned model creation step of creating a learned model using the morphological information regarding the bioparticle and the information regarding the molecule.

### REFERENCE SIGNS LIST

1 Capturing device
11 Linker
12 Amplification sequence part
13 Barcode sequence part
14 UMI unit
15 Molecule capturing sequence part
16 Bioparticle capturing part
17 Collection sequence part
2 Information processing device
21 Processing unit
22 Storage unit
23 User interface unit
24 Output unit
3 Imaging device
4 Fluid control unit
150 Microchip
100 Bioparticle analysis system
101 Surface
102 Analysis substrate
103 Imaging element
104 Light source

1. A bioparticle analysis system comprising:
a capturing device that captures a bioparticle via a bioparticle capturing part on a surface on which a cleavable linker, the bioparticle capturing part, a molecule capturing sequence part, and a barcode sequence part are immobilized via the linker; and
an information processing device that associates morphological information regarding the bioparticle obtained on a basis of captured image information with information regarding a molecule obtained on a basis of a sequence of the barcode sequence part assigned to the molecule derived from the bioparticle captured by the molecule capturing sequence part.

2. The bioparticle analysis system according to claim 1, wherein the information processing device extracts a feature amount on a basis of the captured image information of the bioparticle.

3. The bioparticle analysis system according to claim 1, wherein the barcode sequence part is associated with position information of the surface in advance.

4. The bioparticle analysis system according to claim 2, wherein
an ID number is assigned to the barcode sequence part, and
the information processing device associates the feature amount with the ID number.

5. The bioparticle analysis system according to claim 1, wherein the bioparticle is stimulated by a drug.

6. The bioparticle analysis system according to claim 1, wherein
the information processing device creates a learned model obtained by machine learning, and
the learned model receives input of the morphological information regarding the bioparticle and outputs related molecular information data.

7. The bioparticle analysis system according to claim 6, wherein the information regarding the molecule is estimated from the morphological information regarding the bioparticle using the learned model.

8. The bioparticle analysis system according to claim 1, further comprising an imaging device that images the bioparticle captured on the surface.

9. The bioparticle analysis system according to claim 1, wherein the bioparticle is a cell or a mass of cells.

10. An information processing device that associates morphological information regarding a bioparticle obtained on a basis of captured image information with information regarding a molecule obtained on a basis of a barcode sequence part assigned to the molecule derived from the bioparticle.

11. A bioparticle analysis method comprising:
a capturing step of capturing a bioparticle via a bioparticle capturing part on a surface on which a cleavable linker, the bioparticle capturing part, a molecule capturing sequence part, and a barcode sequence part are immobilized via the linker;
an imaging step of imaging the bioparticle captured on the surface;
a sequence analysis step of analyzing a sequence of the barcode sequence part assigned to a molecule derived from the bioparticle captured by the molecule capturing sequence part; and
an association step of associating morphological information regarding the bioparticle obtained on a basis of captured image information obtained in the imaging step with information regarding the molecule obtained on a basis of the sequence of the barcode sequence part obtained in the sequence analysis step.

12. The bioparticle analysis method according to claim 11, further comprising: a stimulation step of stimulating the bioparticle by a drug.

13. The bioparticle analysis method according to claim 11, further comprising: a learned model creation step of creating a learned model using the morphological information regarding the bioparticle and the information regarding the molecule.
